(19) United States
(12) Patent Application Publication
(10) Pub. No.: US 2003/0113704 A1
(43) Pub. Date: Jun. 19, 2003

(54) Methods for inactivation of pathogens in biological materials

(75) Inventors: Adonis Stassinopoulos, Dublin, CA (US); David Cook, Lafayette, CA (US)

Correspondence Address: John W. Tessman, Cerus Corporation, Suite 300, 2525 Stanwell Drive, Concord, CA, 94520, US

(73) Assignee: Cerus Corporation, Concord, CA

(21) Appl. No.: 10/043,855

(22) Filed: Dec. 21, 2001

Related U.S. Application Data
(60) Provisional application No. 60/257,523, filed on Dec. 21, 2000.

(51) Int. Cl. A01N 1/02; A61K 35/12; C02F 1/68

(52) U.S. Cl. 435/2; 424/520; 210/749

(57) ABSTRACT

Methods are provided for inactivation of pathogens in biomaterials. Pathogen inactivating agents are added to and mixed with biomaterials in an additive solution that is low in chloride and/or hypotonic, resulting in substantial increases in inactivation of bacterial pathogens, particularly Gram negative bacterial pathogens.

### REFERENCE TO RELATED APPLICATIONS

This application claims priority to U.S. Provisional Patent Application Serial No. 60/257,523, filed Dec. 21, 2000; the disclosure of which is hereby incorporated by reference.

### BACKGROUND OF THE INVENTION

Donated blood is used for a variety of different blood products, including packed red blood cells (PRBC), platelet concentrate, and plasma. Blood is also used as the starting material for the purification of a number of different proteins, particularly clotting factors. A serious concern with donated blood is that pathogens such as viruses, bacteria and protozoans can be transmitted via the blood supply, presenting a significant public health issue throughout the world.

Although the blood supply is screened for viral pathogens such as hepatitis B virus (HBV), hepatitis C virus (HCV) and human immunodeficiency virus (HIV), transmission of blood-borne diseases persists. Most screening assays for viruses rely on serum anti virus antibodies, but these antibodies only appear after a lag period of weeks or months after exposure to the virus. The existence of the lag period makes it possible for virus contaminated blood or blood products to pass undetected in screening assays. Bacterial contamination of blood products is particularly problematic due to the potential for bacterial proliferation during storage of the blood or blood product. Additionally, there are currently no licensed tests to screen for bacterial contamination of blood products.

A method of inactivating such pathogens in blood products would be extremely beneficial. In addition to inactivating any virus that is missed by the screening assays and inactivating pathogens for which there is no screening assay, a pathogen inactivation process in blood products could potentially avoid the spread of any emerging pathogens. However, the presence of relatively large and/or labile entities such as red blood cells (RBC), platelets, and enzymes makes pathogen inactivation in blood and blood products particularly challenging. Further complicating efforts to inactivate bacterial pathogens is the necessity for inactivating both Gram negative and Gram positive bacteria. These two classes of bacteria may respond very differently to inactivating agents due to the difference in their physiological characteristics and their membrane composition and structure.

Several methods have been proposed for pathogen inactivation in blood products. The introduction of chemical agents into blood or blood plasma has been suggested to inactivate pathogens prior to clinical use of the blood product. For example, nitrogen mustard, CH₃—N(CH₂CH₂Cl)₂, has been tested for use as a virucidal agent in blood products, but substantial hemolysis was induced at the concentrations necessary to inactivate one of the viruses studied, rendering nitrogen mustard unsuitable for use in blood (LoGrippo et al., Proceedings of the Sixth Congress of the International Society of Blood Transfusion, Bibliotheca Haematologica (Hollander, ed.), 1958, pp. 225-230). A similar approach is presented in U.S. Pat. Nos. 6,093,564 and 6,136,586, which disclose a more selective ethyleneimine oligomer as the inactivating agent. Another approach can be found in U.S. Pat. Nos. 6,093,725 and 6,143,490, which disclose a number of bifunctional compounds comprising a DNA binding portion linked to a DNA modifying portion for use in inactivating pathogens in biological materials such as blood. Unlike nitrogen mustard, the latter approaches are potentially effective in blocking pathogen nucleic acid replication without significantly altering the function of the blood product.

There are also proposed chemical agents that require an external source of activation, for example, photochemical agents that inactivate pathogens upon irradiation with appropriate wavelengths of light. U.S. Pat. No. 5,871,900 discloses psoralens for inactivation of pathogens in blood and blood products. Because psoralens require UVA light to react, they are more effective in those blood products that do not contain red cells, which contribute significant absorption of the UVA wavelengths. Several photochemical approaches to inactivation of pathogens in red cells exist. For example, the use of phthalocyanines or thiazine dyes and visible light has been demonstrated (U.S. Pat. Nos. 5,232,844 and 5,827,644).

Typically, PRBC are prepared in solutions containing citrate, phosphate, glucose and adenine. Such solutions are intended to extend the lifetime of the red cells. See, for example, U.S. Pat. No. 5,250,303. One commonly used solution for RBC storage is ADSOL® solution (available from Fenwal Laboratories), a slightly hypertonic solution containing adenine, mannitol, glucose, and sodium chloride. It has been found that bacterial inactivation with certain chemical agents is sensitive to the additive solution that is used for storing of red cells. Thus, there is a need to find a suitable method of pathogen inactivation, with an appropriate additive solution, which gives optimal bacterial inactivation without substantially affecting the utility of the blood or blood products.

### SUMMARY OF THE INVENTION

The inventors have found that use of certain pathogen inactivating agents in a solution that is low in chloride ions and/or hypotonic (“low chloride/hypotonic solution”) results in a substantial improvement in inactivation of bacterial pathogens, particularly Gram negative bacterial pathogens such as Yersinia enterocolitica, Pseudomonas fluorescens, Serratia marcescens, and Salmonella Typhymurium, without significantly decreasing the level of inactivation of Gram positive bacterial and viral pathogens. Improved inactivation of Gram negative bacterial pathogens is highly desirable, due to the relative resistance of Gram negative bacteria to common pathogen inactivation methods. Yersinia enterocolitica and Pseudomonas fluorescens are of particular concern in PRBC as they are known contaminants resulting in bacterial sepsis after red cell transfusion and are able to grow at 4° C., the storage temperature of red cells (Gottlieb, Anaesth. Intens. Care 21:20 (1993)). While it is important to improve the inactivation of Gram negative bacteria, the methods of the present invention may also result in improved inactivation of certain strains of Gram positive bacteria.

Thus, the invention provides new methods and compositions for the inactivation of viral and bacterial, particularly Gram negative bacterial, pathogens in biological materials such as blood, blood products, and other blood-derived materials such as purified clotting factors. The methods of the invention utilize a pathogen inactivating agent (generally a DNA modifying compound) in an additive solution that is low in chloride and/or hypotonic. The pathogen inactivation results in a biological material that remains suitable for its intended use.

One composition of the present invention comprises a solution for the inactivation of a Gram negative bacteria comprising (a) an additive solution wherein chloride ion, if present, is at a concentration of less than about 10 mM, (b) a biological material suspected of containing a Gram negative bacteria; and (c) a pathogen inactivation compound in an amount sufficient to inactivate at least 1 log of the Gram negative bacteria. In another embodiment, the additive solution is essentially free of chloride ions. In another embodiment, the additive solution is hypotonic. In another embodiment, the Gram negative bacteria is selected from the group consisting of Yersinia enterocolitica, Pseudomonas fluorescens, Serratia marcescens, Salmonella Typhymurium, Salmonella choleraesuis, Escherichia coli K12, Pseudomonas aeruginosa, and Serratia liquifaciens. In another embodiment, the Gram negative bacteria is selected from the group consisting of Yersinia enterocolitica, Pseudomonas fluorescens, Serratia marcescens, and Salmonella Typhymurium. In a preferred embodiment, the Gram negative bacteria is Yersinia enterocolitica. In another embodiment, the biological material is a blood product, preferably comprising red blood cells. In another embodiment, the pathogen inactivation compound is more reactive at physiological pH than at a pH of about 4. A preferred inactivation compound inactivates more of the Gram negative bacteria using an additive solution which is essentially free of chloride ions as compared to the inactivation obtained using a similar additive solution with a chloride ion concentration of greater than about 10 mM.

Another composition of the present invention comprises a solution for the inactivation of a Gram negative bacteria comprising (a) an additive solution lacking chloride ions, (b) a biological solution comprising red blood cells suspected of containing a Gram negative bacteria; and (c) a pathogen inactivation compound in an amount sufficient to inactivate at least 1 log of Yersinia enterocolitica bacteria, wherein the amount of inactivation is at least 1 log greater than the inactivation of a similar composition in which the additive solution contains greater than about 10 mM chloride ions.

The present invention also provides a method of inactivating a Gram negative bacteria in a biological material suspected of containing the Gram negative bacteria, the method comprising (a) contacting the biological material with an additive solution comprising a chloride concentration of less than about 10 mM, (b) contacting the biological material with a pathogen inactivation compound in an amount sufficient to inactivate at least 1 log of the Gram negative bacteria, and (c) incubating the biological material contacted with the additive solution and the pathogen inactivation compound for sufficient time to inactivate at least 1 log of the Gram negative bacteria. In another embodiment, the additive solution is essentially free of chloride ions. In another embodiment, the additive solution is hypotonic. In another embodiment, the Gram negative bacteria is selected from the group consisting of Yersinia enterocolitica, Pseudomonas fluorescens, Serratia marcescens, Salmonella Typhymurium, Salmonella choleraesuis, Escherichia coli K12, Pseudomonas aeruginosa, and Serratia liquifaciens. In another embodiment, the Gram negative bacteria is selected from the group consisting of Yersinia enterocolitica, Pseudomonas fluorescens, Serratia marcescens, and Salmonella Typhymurium. In a preferred embodiment, the Gram negative bacteria is Yersinia enterocolitica. In another embodiment, the biological material is a blood product, preferably comprising red blood cells. In another embodiment, the pathogen inactivation, compound is more reactive at physiological pH than at a pH of about 4.

In another embodiment, the present invention provides a method of inactivating a Gram negative bacteria in a biological material suspected of containing the Gram negative bacteria comprising (a) contacting the biological material with a first additive solution which is essentially chloride free, (b) contacting the biological material with a pathogen inactivation compound in an amount sufficient to inactivate at least 1 log of the Gram negative bacteria, wherein the pathogen inactivation compound has a greater inactivation efficiency against Yersinia enterocolitica when used with said first additive solution than when used with a second additive solution, said second additive solution comprising at least about 10 mM chloride ion; and (c) incubating the biological material contacted with the first additive solution and the pathogen inactivation compound for sufficient time to inactivate at least 1 log of the Gram negative bacteria. In another embodiment, the additive solution is hypotonic. In another embodiment, the Gram negative bacteria is selected from the group consisting of Yersinia enterocolitica, Pseudomonas fluorescens, Serratia marcescens, Salmonella Typhymurium. Salmonella choleraesuis, Escherichia coli K12, Pseudomonas aeruginosa, and Serratia liquifaciens. In another embodiment, the Gram negative bacteria is selected from the group consisting of Yersinia enterocolitica, Pseudomonas fluorescens, Serratia marcescens, and Salmonella Typhymurium. In a preferred embodiment, the Gram negative bacteria is Yersinia enterocolitica. In another embodiment, the biological material is a blood product, preferably comprising red blood cells. In another embodiment, the pathogen inactivation compound is more reactive at physiological pH than at a pH of about 4.

In another embodiment, the present invention provides a method of inactivating a Gram negative bacteria in a biological material suspected of containing the Gram negative bacteria comprising (a) contacting the biological material with a pathogen inactivation compound in an amount sufficient to inactivate at least 1 log of the Gram negative bacteria and an additive solution comprising a chloride concentration of less than about 10 mM, and (b) incubating the biological material contacted with the additive solution and the pathogen inactivation compound for sufficient time to inactivate at least 1 log of the Gram negative bacteria. In another embodiment, the additive solution is essentially free of chloride ions. In another embodiment, the additive solution is hypotonic. In another embodiment, the Gram negative bacteria is selected from the group consisting of Yersinia enterocolitica, Pseudomonas fluorescens, Serratia marcescens, Salmonella Typhymurium, Salmonella choleraesuis, Escherichia coli K12, Pseudomonas aeruginosa, and Serratia liquifaciens. In another embodiment, the Gram negative bacteria is selected from the group consisting of Yersinia enterocolitica, Pseudomonas fluorescens, Serratia marcescens, and Salmonella Typhymurium. In a preferred embodiment, the Gram negative bacteria is Yersinia enterocolitica. In another embodiment, the biological material is a blood product, preferably comprising red blood cells. In another embodiment, the pathogen inactivation compound is more reactive at physiological pH than at a pH of about 4.

In another embodiment, the present invention provides a method of inactivating a Gram negative bacteria in a biological material suspected of containing the Gram negative bacteria comprising (a) contacting the biological material with a first additive solution which is essentially chloride free and a pathogen inactivation compound in an amount sufficient to inactivate at least 1 log of the Gram negative bacteria, wherein the pathogen inactivation compound has a greater inactivation efficiency against Yersinia enterocolitica when used with said first additive solution than when used with a second additive solution, said second additive solution comprising at least about 10 mM chloride ion; and (b) incubating the biological material contacted with the first additive solution and the pathogen inactivation compound for sufficient time to inactivate at least 1 log of the Gram negative bacteria. In another embodiment, the additive solution is hypotonic. In another embodiment, the Gram negative bacteria is selected from the group consisting of Yersinia enterocolitica, Pseudomonas fluorescens, Serratia marcescens, Salmonella Typhymurium, Salmonella choleraesuis, Escherichia coli K12, Pseudomonas aeruginosa, and Serratia liquifaciens. In another embodiment, the Gram negative bacteria is selected from the group consisting of Yersinia enterocolitica, Pseudomonas fluorescens, Serratia marcescens, and Salmonella Typhymurium. In a preferred embodiment, the Gram negative bacteria is Yersinia enterocolitica. In another embodiment, the biological material is a blood product, preferably comprising red blood cells. In another embodiment, the pathogen inactivation compound is more reactive at physiological pH than at a pH of about 4.

Another method of the present invention comprises a method of inactivating a Gram negative bacteria in a red blood cell composition suspected of containing Yersinia enterocolitica comprising (a) contacting the red cell composition with a first additive solution lacking chloride ions, (b) contacting the red cell composition with a pathogen inactivation compound in an amount sufficient to inactivate at least 1 log of the Yersinia enterocolitica, wherein the pathogen inactivation compound has a greater inactivation efficiency against Yersinia enterocolitica when used with the first additive solution than when used with a second additive solution, said second additive solution comprising at least about 10 mM chloride ion, and (c) incubating the biological material contacted with the first additive solution and the pathogen inactivation compound for sufficient time to inactivate at least 1 log of the Gram negative bacteria. Preferably, the inactivation of Yersinia enterocolitica using the first additive solution is at least 1 log better than the inactivation when using the second additive solution.

Generally, a biomaterial, such as whole blood, PRBC, platelet concentrate plasma, or purified protein (e.g., purified clotting factors), is treated such that the material is in a solution or suspension in a low chloride/hypotonic solution (e.g., by diluting, dissolving, resuspending, or dialyzing with an additive solution which is low in chloride and/or hypotonic). A pathogen inactivating agent is added to the biomaterial and incubated. If necessary, the pathogen inactivating agent is activated before, during or after addition to the biomaterial. In a preferred embodiment, the pathogen inactivating agent does not require an external source of energy, e.g. light energy, to be activated. A suitable quenching agent may optionally be added to the incubation mixture prior to, simultaneously with, or after the addition of the pathogen inactivating agent.

In certain embodiments, the pathogen inactivating agent comprises a functional unit that is an alkylating agent. Preferably, the functional unit is selected from the group consisting of mustard groups, mustard intermediates, mustard group equivalents, epoxides, aldehydes, and formaldehyde synthons. The present invention contemplates an embodiment wherein the pathogen inactivating agent is β-alanine, N-(acridin-9-yl), 2-[bis(2-chloroethyl)amino]ethyl ester, which comprises a nucleic acid binding portion in addition to an alkylating agent.

In other embodiments the low chloride/hypotonic solution is either Erythrosol™, Solution 2, CPD, or CPDA-1. Erythrosol™ consists of 25.0 mM sodium citrate, 16.0 mM disodium phosphate, 4.4 mM monosodium phosphate, 1.5 mM adenine, 39.9 mM mannitol, and 45.4 mM dextrose. Solution 2 consists of 21.9 mM sodium citrate, 31.5 mM disodium phosphate, 18.0 mM monosodium phosphate, 2.44 mM adenine, 67.2 mM mannitol, and 110 mM dextrose. CPD consists of 89.4 mM sodium citrate, 17.0 mM citric acid, 142.0 mM dextrose, and 18.5 mM monosodium phosphate. CPDA-1 consists of 89.4 mM sodium citrate, 17.0 mM citric acid, 177.0 mM dextrose, and 18.5 mM monosodium phosphate.

### DETAILED DESCRIPTION OF THE INVENTION

Use of a pathogen inactivating agent in low chloride/hypotonic solution results in a substantial improvement in inactivation of Gram negative bacterial pathogens such as Yersinia enterocolitica, Pseudomonas fluorescens, Serratia marcescens, Salmonella Typhymurium, Salmonella choleraesuis, Escherichia coli K12, Pseudomonas aeruginosa, and Serratia liquifaciens without significantly decreasing the level of inactivation of Gram positive bacterial and viral pathogens. Improved inactivation of Gram negative bacterial pathogens is highly desirable, due to the relative resistance of Gram negative bacteria to common pathogen inactivation methods. The inventors have surprisingly and unexpectedly found that inactivation of Gram negative bacteria in a biological material greatly depends upon the additive solution in which the inactivation takes place. The use of low chloride or essentially chloride free additive solutions, particularly hypotonic low chloride or essentially chloride free additive solutions, results in substantial increases in inactivation of Gram negative bacteria without significantly decreasing the inactivation of Gram positive bacterial and viral pathogens. The use of the additive solutions of the present invention may also result in a substantial increase in the inactivation of certain strains of Gram positive bacteria, such as Staphylococcus epidermidis.

Definitions

The term “aqueous mixture” refers to a mixture that contains water as a solvent. An aqueous mixture may also contain solvents other than water. A preferred aqueous mixture contains water as the primary solvent. An aqueous mixture may be an aqueous solution (e.g., containing solutes dissolved in the water, such as a red cell storage solution), a suspension (e.g., containing non-dissolved substances in the solvent, such as a suspension of red blood cells), or have the characteristics of both a solution and a suspension (e.g., containing both dissolved solutes and non-dissolved substances, such as a suspension of red blood cells in a storage solution).

A “vessel” is a container that is capable of holding a liquid mixture. Acceptable vessels may be constructed with rigid walls, such as beakers, flasks, tanks, and the like, or they may have flexible walls, such as ‘blood bags’ (e.g., flexible plastic bags made of materials such as EVA and/or PVC having one or more ports for access to the interior of the bag).

A pathogen is considered “inactivated” when its ability to reproduce under appropriate conditions is severely or substantially hampered (e.g., when a bacterial pathogen is unable to form colonies visible to the unaided eye in a colony formation assay). Cellular pathogens such as bacteria, fungi, and molds are considered inactivated when they are severely hampered from reproducing under physical and nutritional conditions that would normally permit reproduction (e.g., in the presence of the appropriate nutrients, temperature, dissolved gases and the like required by the particular pathogen). Non-cellular pathogens such as viruses are considered inactivated when they are severely hampered from reproducing when placed under physical and nutritional conditions and in the presence of a host cell which would normally support reproduction (e.g., in the presence of a permissive host cell which is in the presence of the appropriate nutrients, temperature, dissolved gases and the like required by the particular pathogen).

Measurement of pathogen inactivation is expressed as the negative logarithm of the fraction of remaining pathogens capable of reproducing. For example, if a compound at a certain concentration renders 90% of the pathogens in a material incapable of reproduction, 10% or one-tenth (0.1) of the pathogens remain capable of reproduction. The negative logarithm of 0.1 is 1, and that concentration of that compound is said to have inactivated the pathogens present by 1 log, or the compound is said to have 1 log inactivation at that concentration. The log inactivation can also be viewed as the comparison of pathogen titer in a control sample to a treated sample, where the log of the ratio of control titer to titer remaining after inactivation represents the log inactivation. For example, if a control titer measures 10⁷ (i.e. a 10⁷ dilution of the solution results in no detection of the pathogen where a 10 dilution results in detection) and a treated sample titer measures 10 (i.e. a 102 dilution of the solution results in no detection of the pathogen where a 10 dilution results in detection), the resulting level of inactivation is 5 logs.

As used herein, the term “hypotonic” refers to a solution having a lower osmolarity than cellular cytoplasm, particularly Gram negative bacterial cytoplasm (i.e., a solution that induces movement of water into Gram negative bacteria suspended in the solution). A hypotonic solution is also one that has an osmolarity of less than about 325 or 300 milliosmolar. The osmolarity is derived by adding the molarities of all ions and non-ionizable elements/compounds in solution. In certain solutions, such as a suspension of red cells, the effective osmolarity may be derived by adding the molarities of all ions and non-ionizable elements/compounds except for those ions/elements that penetrate the cell membrane and readily equilibrate, such as dextrose. The osmolarity of a solution can be readily measured by methods known to one skilled in the art. Preferably, a hypotonic solution for use in the instant invention is also pH buffered to a physiological pH, generally about pH 6.2 to 8.0, more preferably about pH 7.2 to 7.8. If a hypotonic solution is pH buffered, it may be referred to as a “hypotonic buffer”.

The term “low chloride” refers to a solution that is essentially free of chloride ions. Preferably, a low chloride solution has less than about 10 mM free chloride ions, although lower levels of free chloride ions (e.g., less than about 5 mM or less than about 1 mM) are preferred. The term “low chloride solution” includes solutions that are essentially chloride free. “Low chloride solutions” include solutions which are pH buffered; such solutions may alternately be referred to as “low chloride buffers”. Solutions that are essentially chloride free are preferably free of chloride ions. Such solutions may contain very low levels of chloride ion, for example, in samples where a small amount of a compound is added which has chloride as a counter ion. For example, pathogen inactivation compounds of the present invention may be chloride salts which, when added to a solution, would result in low chloride concentrations. Such solutions would be considered essentially chloride free and are considered “low chloride solutions”. Preferably, a low chloride solution for use in the instant invention is pH buffered to a physiological pH, generally about pH 6.2 to 8.0, more preferably about pH 7.2 to 7.8. Low chloride solutions may also be generated by incubating a solution free of chloride ions with with cells which contain physiological amounts of chloride ions. It is expected that the choride ions will traverse the cell membrane, thereby generating the low chloride solution.

As used herein, the term “biological material” or “biomaterial” refers to a material originating from a biological organism of any type. Examples of biological materials include, but are not limited to, whole blood, blood products including packed red blood cells (PRBC), platelets, fresh or frozen plasma, plasma fraction products, (e.g. antihemophilic factor (Factor VIII), Factor IX and Factor IX complex, fibrinogens, Factor XIII, prothrombin and thrombin, immunoglobulins (such as IgG, IgA, IgD, IgE and IgM and fragments thereof), and albumin, serum, interferons, lymphokines, vaccines, recombinant DNA produced proteins, oligopeptide ligands, milk, clinical samples such as urine, sweat, sputum, feces and spinal fluid, cellular and tissue extracts from vertebrate cells or tissues, and any other substance having its origin in a biological organism, as well as synthetic blood, synthetic blood products and blood product storage media. Biological materials also include synthetic material incorporating a substance having its origin in a biological organism, such as a vaccine preparation comprised of alum and a pathogen (the pathogen, in this case, being the substance having its origin in a biological organism), a sample prepared for analysis which is a mixture of blood and analytical reagents, cell culture medium, cell cultures, viral cultures, and other cultures derived from a living organism, as well as purified and partially purified preparations derived from biological materials, such as clotting factors. Biological materials also include vertebrate proteins and structural and functional equivalents thereof produced using recombinant technology (e.g., murine antibodies and chimeric or humanized derivatives thereof produced in bacterial host cells).

The term “blood product” refers to all formulations of the fluid and/or associated cellular elements and the like (such as erythrocytes, leukocytes, platelets, etc.) that pass through a vertebrate organism's circulatory system; blood products include, but are not limited to, packed red blood cells (PRBC), platelet mixtures, serum, and plasma. Blood products include “purified blood products”, which are fractionated materials derived from a blood product, or synthetic or recombinant equivalents thereof. Purified blood products include clotting factors, growth factors, protein hormones, albumin, immunoglobins, and the like, as well as synthetic or recombinant versions thereof. The term “platelet mixture” refers to one type of blood product wherein the cellular element is primarily or only platelets. A platelet concentrate (PC) is one type of platelet mixture where the platelets are associated with a smaller than normal portion of plasma. A synthetic media may make up that volume normally occupied by plasma; for example, a platelet concentrate may entail platelets suspended in 35% plasma/65% synthetic media. The synthetic media might also comprise phosphate.

“Pathogen” is defined as any nucleic acid containing agent capable of causing disease in a human, other mammals, or vertebrates. Examples include microorganisms such as unicellular or multicellular microorganisms. Examples of pathogens are bacteria, viruses, protozoa, fungi, yeasts, molds, and mycoplasma that cause disease in humans, other mammals, or vertebrates. The genetic material of the pathogen may contain DNA or RNA, and the genetic material may be present as single-stranded or double-stranded nucleic acid. The nucleic acid of the pathogen may be in solution, intracellular, extracellular, or bound to cells.

The terms “Gram positive bacteria” and “Gram negative bacteria” refer to two distinct classes of bacteria. Gram positive bacteria are those bacterial species that lack an outer membrane while Gram negative bacteria have an outer membrane surrounding the cell wall. Gram positive or negative bacteria are readily identified by methods known to one skilled in the art. Examples of Gram negative bacteria include Yersinia enterocolitica, Pseudomonas fluorescens, Serratia marcescens, Salmonella typhymurium, Salmonella choleraesuis, Escherichia coli K12, Pseudomonas aeruginosa, and Serratia liquifaciens. Gram positive bacteria include Staphylococcus aureus, Staphylococcus epidermidis, Deinococcus radiodurans, Listeria monocytogenes, and Bacilus subtilis.

As used herein, the term “pathogen inactivating agent” refers to chemical compounds that significantly inhibit the reproduction of pathogens and/or can render pathogens incapable of reproducing. Preferred pathogen inactivating agents can covalently modify nucleic acid, thereby inhibiting and/or blocking nucleic acid replication. Examples of pathogen inactivating agents for use in the instant invention include nucleic acid alkylators such as bifunctional compounds possessing a nucleic acid binding portion linked to an effector portion which covalently modifies DNA, such as those described in U.S. Pat. Nos. 6,093,725 and 6,143,490.

As used herein, the term “comprising” and its cognates are used in their inclusive sense; that is, equivalent to the term “including” and its corresponding cognates.

Additive Solutions

As used herein, the term “additive solution” refers to a solution in which the biological materials are diluted, resuspended or dissolved during pathogen inactivation. Additive solutions in accordance with the invention are low in chloride or essentially chloride free and/or hypotonic. Additionally, additive solutions are preferably pH buffered to a physiologically-acceptable pH, such as from about pH 6.8 to 8.0, more preferably to about pH 7.2 to 7.8. The formulation of a commonly used red blood cell (RBC) storage solution (Adsol) is compared with two exemplary additive solutions (ErythroSol™ and Solution 2) in Table 1.

TABLE 1

| | | | Solution 2 |
|---|---|---|---|
| Ingredients | Adsol (mM) | Erythrosol (mM) | (mM) |
| Sodium Chloride | 154.0 | 0 | 0 |
| Sodium Citrate | 0 | 25.0 | 21.9 |
| Disodium Phosphate | 0 | 16.0 | 31.5 |
| Monosodium Phosphate | 0 | 4.4 | 18.0 |
| Adenine | 2.0 | 1.5 | 2.44 |
| Mannitol | 41.2 | 39.9 | 67.2 |
| Dextrose | 111.0 | 45.4 | 110 |
| Osmolarity | slightly | hypotonic | hypotonic |
| | hypertonic | | |

Where the additive solution is a low chloride solution, the additive solution comprises less than about 10 mM free chloride ions, more preferably less than about 5 mM or 1 mM free chloride ions. Another preferred additive solution is a chloride free additive solution. A chloride free additive solution contains no added chloride ions (i.e., contains no salts of hydrochloric acid or chloride salts of bases). The additive solution may be used to dissolve a dried biomaterial such as a lyophilized protein, to resuspend a particulate biomaterial such as PRBC or packed platelets, or to alter the ionic content of a biomaterial by, for example, dilution or dialysis of whole blood. Because the biomaterial may contain free chloride ions (or release chloride ions after exposure to the additive solution), the biomaterial/additive solution combination (i.e., the product of dissolving, resuspending, diluting or dialyzing the biomaterial with the additive solution) may have a higher free chloride ion concentration than the additive solution alone. To further reduce the amount of chloride ion, the blood product my be washed with more than one aliquot of the chloride free solution.

In one embodiment of the present invention, the additive solution is hypotonic, such that it will induce the movement of water into the intracellular compartment of cells in additive solution. A hypotonic additive solution is less than about 325 mOsmolar, more preferably less than about 300 mOsmolar. A hypotonic solution may be hypotonic due to the total ion and solute concentrations. Alternatively, a solution may be effectively hypotonic when the formal tonicity based on the ion and solute concentrations is above 325 mOsmolar but some of the components readily traverse the cell membranes. This my result in an extracellular medium which is effectively hypotonic. Such solutions, in which the effective hypotonicity is based on ion and solute concentrations of those ions and solutes that do not traverse the cell membranes, are encompassed by the present invention.

In another embodiment, the additive solutions may be pH buffered. pH buffering is generally accomplished by adding one or more salts of acids, such as sodium or potassium salts of phosphate, acetate, citrate, carbonate, and the like. Preferably, a pH buffered additive solution is buffered to a physiologically compatible pH, generally from about pH 6.8 to 8.0, more preferably about pH 7.2 to 7.8.

A preferred additive solution of the present invention is a solution of suitable chloride concentration and/or hypotonicity such that the inactivation of Yersinia enterocolitica in a composition comprising red blood cells using β-alanine, N-(acridin-9-yl), 2-[bis(2-chloroethyl)amino]ethyl ester is improved when compared to a composition in which the preferred additive solution is replaced by Adsol or a solution similar to Adsol. This is demonstrated for a solution similar to Erythrosol in Example 2. The additive solution may increase the inactivation by at least 1 log, preferably at least 2 logs and more preferably at least 3 logs more than the inactivation seen when Adsol is used in PRBC under the conditions of Example 2.

In preferred embodiments, additive solutions of the present invention comprise a sodium chloride concentration of 0 to about 10 mM. Such additive solutions may further comprise sodium citrate, disodium phosphate, monosodium phosphate, adenine and mannitol. In some embodiments, the additive solutions may contain dextrose. In other embodiments, dextrose is added to the red cell composition separately. In an embodiment of the present invention, the additive solution comprises 0 to about 10 mM sodium chloride, about 20-30 mM sodium citrate, about 10-35 mM disodium phosphate, about 4-18 mM monosodium phosphate, about 1-3 mM adenine, and about 35-70 mM mannitol. Additionally, the composition may further comprise about 0-110 mM dextrose. In an embodiment of the present invention, the additive solution comprises about 25 mM sodium citrate, about 16 mM disodium phosphate, about 4.4 mM monosodium phosphate, about 1.5 mM adenine, about 39.9 mM mannitol and about 45.4 mM dextrose. In another embodiment, the additive solution comprises about 26.6 mM sodium citrate, about 17 mM disodium phosphate, about 4.7 mM monosodium phosphate, about 1.6 mM adenine and about 42.5 mM mannitol.

Pathogen Inactivating Agents

The present invention utilizes chemical compounds that can covalently modify nucleic acid, thereby blocking or inhibiting nucleic acid replication, resulting in inactivation of pathogens such as viruses and bacteria. Preferred pathogen inactivating agents for the present invention are activated by an increase or maintenance of the pH of their environment to about physiological pH. Such pathogen inactivating agents exhibit increased reactivity with the nucleic acid at higher pH in a pH range of about 3 to about 8 as measured at room temperature. Such agents are sensitive to small changes in the pH such that intracellular pH changes in Gram negative bacteria will affect the level of inactivation of these bacteria.

One group of preferred pathogen inactivating agents are compounds that have a nucleic acid binding portion and an effector portion linked to each other via covalent bonds. “The nucleic acid binding portion” is a portion that binds non-covalently to a nucleic acid biopolymer such as DNA or RNA, while the “effector portion” is a portion that reacts with the nucleic acid by a mechanism that forms a covalent bond with the nucleic acid. The anchor-effector arrangement enables the pathogen inactivating agents to be targeted to nucleic acid (due to the anchor's binding ability). This brings the effector into proximity for reaction with the nucleic acid, thereby causing a preferential reactivity with nucleic acids as compared to components (i.e., proteins). Another preferred group of pathogen inactivating agents comprise a nucleic acid binding portion and an effector portion covalently linked via a frangible linker. A “frangible linker” is a portion that serves to covalently link the anchor and effector, and which will degrade under certain conditions so that the anchor and effector are no longer linked covalently, preferably after the effector portion has reacted with the nucleic acid.

A wide variety of groups are available for use as the nucleic acid binding portions, linkers, and effector portions. Examples of the binding portion groups which can be used in the pathogen inactivation agents include, but are not limited to, intercalators, minor groove binders, major groove binders, molecules which bind by electrostatic interactions such as polyamines, and molecules which bind by sequence specific interactions. The following is a non-limiting list of possible nucleic acid binding portions: acridines (and acridine derivatives, e.g. proflavine, acriflavine, diacridines, acridones, benzacridines, quinacrines), actinomycins, anthracyclinones, rhodomycins, daunomycin, thioxanthenones (and thioxanthenone derivatives, e.g. miracil D), anthramycin, mitomycins, echinomycin (quinomycin A), triostins, ellipticine (and dimers, trimers and analogs thereof), norphilin A, fluorenes (and derivatives, e.g. flourenones, fluorenodiamines), phenazines, phenanthridines, phenothiazines (e.g., chlorpromazine), phenoxazines, benzothiazoles, xanthenes and thioxanthenes, anthraquinones, anthrapyrazoles, benzothiopyranoindoles, 3,4-benzopyrene, 1-pyrenyloxirane, benzanthracenes, benzodipyrones, quinolines (e.g., chloroquine, quinine, phenylquinoline carboxamides), furocoumarins (e.g., psoralens and isopsoralens), ethidium, propidium, coralyne, and polycyclic aromatic hydrocarbons and their oxirane derivatives; distamycin, netropsin, other lexitropsins, Hoechst 33258 and other Hoechst dyes, DAPI (4′,6-diamidino-2-phenylindole), berenil, and triarylmethane dyes; aflatoxins; spermine, spermidine, and other polyamines; and nucleic acids or analogs which bind by sequence specific interactions such as triple helix formation, D-loop formation, and direct base pairing to single stranded targets. Derivatives of these compounds are also non-limiting examples of nucleic acid binding portions, where a derivative of a pathogen inactivation agent includes, but is not limited to, a compound which bears one or more substituents of any type at any location, oxidation or reduction products of the compound, etc.

Preferred pathogen inactivating agents useful in the present invention comprise as nucleic acid binding portions acridine compounds, acridine dyes, and acridine derivatives. The terms “acridine compound,” “acridine dyes,” and the like refer to a chemical compound containing the tricyclic structure of acridine (dibenzo[b, e]pyridine; 10-azanthracene). Acridines are frequently obtained from coal tar and are used in the manufacture of dyes and antiseptics. The compounds have an affinity for (and can bind) to nucleic acids non-covalently through intercalation. The term “aminoacridine” refers to those acridine compounds with one or more nitrogen-containing functional groups. Examples of aminoacridines include 9-amino acridine; β-alanine, N-(acridin-9-yl), 2-[bis(2-chloroethyl)amino]ethyl ester; and acridine orange.

Examples of frangible linkers which can be part of pathogen inactivating agents useful in the invention are, but are not limited to, compounds which include functional groups such as ester (where the carbonyl carbon of the ester is between the anchor and the sp3 oxygen of the ester; this arrangement is also called “forward ester”), “reverse ester” (where the sp3 oxygen of the ester is between the anchor and the carbonyl carbon of the ester), thioester (where the carbonyl carbon of the thioester is between the anchor and the sulfur of the thioester, also called “forward thioester”), reverse thioester (where the sulfur of the thioester is between the anchor and the carbonyl carbon of the thioester, also called “reverse thioester”), forward and reverse thionoester, forward and reverse dithioic acid, sulfate, forward and reverse sulfonates, phosphate, and forward and reverse phosphonate groups. “Thioester” designates the —(═O)—S— group; “thionoester” designates the —C(═S)—O— group, and “dithioic acid” designates the —C(═S)—S— group. The frangible linker also may include an amide, where the carbonyl carbon of the amide is between the anchor and the nitrogen of the amide (also called a “forward amide”), or where the nitrogen of the amide is between the anchor and the carbonyl carbon of the amide (also called a “reverse amide”). For groups which can be designated as “forward” and “reverse”, the forward orientation is that orientation of the functional groups wherein, after hydrolysis of the functional group, the resulting acidic function is covalently linked to the anchor portion and the resulting alcohol or thiol function is covalently linked to the effector portion. The reverse orientation is that orientation of the functional groups wherein, after hydrolysis of the functional group, the resulting acidic function is covalently linked to the effector portion and the resulting alcohol or thiol function is covalently linked to the anchor portion.

The frangible linker, such as an amide portion, also may be capable of degrading under conditions of enzymatic degradation, by endogenous enzymes in the biological material being treated, or by enzymes added to the material.

Examples of the effector portions which can be used in pathogen inactivating agents useful in the invention are, but are not limited to, mustard groups, mustard intermediates, mustard group equivalents, epoxides, aldehydes, formaldehyde synthons, and other alkylating and cross-linking agents.

Mustard groups are defined as including mono or bis haloethylamine groups, and mono haloethylsulfide groups. Mustard group equivalents are defined by groups that react by a mechanism similar to the mustards (that is, by forming an aziridinium intermediate, or by having or by forming an aziridine ring, which can react with a nucleophile), such as aziridine derivatives, mono or bis-(mesylethyl)amine groups, mono mesylethylsulfide groups, mono or bis tosylethylamine groups, and mono tosylethylsulfide groups. Formaldehyde synthons are defined as any compound that breaks down to formaldehyde in aqueous solution, including hydroxymethylamines such as hydroxymethylglycine. Examples of formaldehyde synthons are given in U.S. Pat. No. 4,337,269 and in International Patent Application WO 97/02028. While the invention is not limited to pathogen inactivating agent, the effector groups, which are, or are capable of forming an electrophilic group, such as a mustard group, are believed to react with and form a covalent bond to nucleic acid.

The effector groups are not limited to mustards. It is believed that mustards can form reactive intermediates such as aziridinium or aziridine complexes and sulfur analogs of these complexes. The present invention also contemplates the use of pathogen inactivating agents with functional groups that are the equivalent of mustards, such as epoxides.

A preferred pathogen inactivating agent of the invention is β-alanine, N-(acridin-9-yl), 2-[bis(2-chloroethyl)amino]ethyl ester, as shown in the formula below:

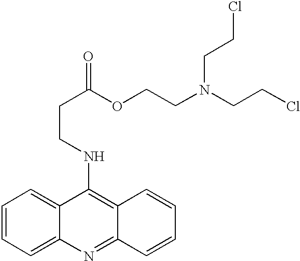

Other exemplary pathogen inactivating agents of the invention are described in U.S. Pat. Nos. 5,691,132, 6,093,725 and 6,143,490, hereby incorporated by reference.

Pathogen inactivating agents are often used in conjunction with a quencher, which is a chemical compound that reduces undesired side reactions of the pathogen inactivating agents in biological materials. Quenching agents useful in the instant invention are disclosed in U.S. patent application Ser. No. 09/100,776, published as International Patent Application No. WO 99/34839. In general, compounds that can quench undesired side reactions of a pathogen inactivating agent include nucleophilic functional groups such as thiols, thioacids, dithoic acids, phosphates, thiophosphates and amines. Exemplary quenchers include glutathione, N-acetylcysteine, cysteine, thiosulfate, mercaptoethanesulfonate salts, and dimercaprol. In a preferred embodiment, the quencher is glutathione.

A suitable pathogen inactivating agent of the present invention is a compound that shows a higher level of inactivation of Yersinia enterocolitica in a composition comprising red blood cells using Erythrosol or solutions similar to Erythrosol as the additive solution as compared to a composition in which the preferred additive solution is replaced by Adsol or a solution similar to Adsol. This is demonstrated for P-alanine, N-(acridin-9-yl), 2-[bis(2-chloroethyl)amino]ethyl ester in Example 2. With a suitable pathogen inactivation compound, Yersinia inactivation using Erythrosol in PRBC should increase by at least 1 log, preferably at least 2 logs and more preferably at least 3 logs than when Adsol is used under the conditions of Example 2.

Inactivation of Pathogens

The biological material is dissolved, resuspended, diluted, or dialyzed with an additive solution in accordance with the invention. A pathogen inactivating agent is added to the biological material or the additive solution, or included with the additive solution used for dissolving, resuspending, diluting or dialyzing the biological material.

The biological material is dissolved, resuspended, diluted or dialyzed with an additive solution of the invention (with or without the pathogen inactivating agent and optional quenching agent) using any appropriate method known in the art. For example, where the biological material is a blood product such as PRBC or platelets, manipulations of the biological material are usually carried out in “blood bags”, and solutions are introduced or removed using tubing attached to one or more ports on the bag. For acellular biological materials such as extracts, and purified proteins and clotting factors, it is generally more convenient to manipulate the materials in ‘batch’ format, using large vessels, pumps, centrifuges, etc., as are commonly used in the art.

The pathogen inactivating agent is added in an amount effective to inactivate pathogens, normally in an amount which is sufficient to inactivate at least about 1, 2, 3, or 4 logs, or, for example, at least about 3 to 6 logs of a pathogen in the sample. Typical concentrations of pathogen inactivating agent for the treatment of biological materials such as blood products are on the order of about 0.1 μM to 5 mM, or about 1 μM to about 1 mM, or about 10 μM to about 750 μM, for example about 300 μM. In certain embodiments, the pathogen inactivating agent produces at least 1 log inactivation at a concentration of no greater than about 500 μM, more preferably at least 3 logs inactivation at no greater than 500 μM concentration. In another non-limiting example, the pathogen inactivating agent will accomplish at least 1 log inactivation, and preferably at least 6 logs inactivation at a concentration of about 0.1 μM to about 3 mM

If a quenching agent is used in the methods of the invention, the quenching agent is added in an amount effective to reduce damage and/or modification of the biological material. Quenching agents suitable for use in the instant invention are disclosed in U.S. patent application Ser. No. 09/110,776 (published as International Patent Application No. WO 99/34839), and include compounds which include nucleophilic groups, or other groups that react with electrophilic groups. Mixtures of quenching compounds also may be used. Exemplary nucleophilic groups include thiol, thioacid, dithioic acid, thiocarbamate, dithiocarbamate, amine, phosphate, and thiophosphate groups. The quencher may be, or contain, a nitrogen heterocycle such as pyridine. The quencher can be a phosphate containing compound such as glucose-6-phosphate. The quencher also can be a thiol containing compound, including, but not limited to, glutathione, cysteine, N-acetylcysteine, mercaptoethanol, dimercaprol, mercaptan, mercaptoethanesulfonic acid and salts thereof, e.g., MESNA, homocysteine, aminoethane thiol, dimethylaminoethane thiol, dithiothreitol, and other thiol containing compounds. The quenchers also can be in the form of a salt, such as sodium or hydrochloride salt. A preferred quenching agent is glutathione. If glutathione is included in the reaction, it is added at about a 1:1 to 100:1 molar ratio with the pathogen inactivating agent, more preferably about 5: 1 to 20:1 or about 10:1 molar ratio.

After or concurrent with the addition of the pathogen inactivating agent and optional quenching agent, the biomaterial and pathogen inactivating agent are mixed. Mixing may be accomplished by any convenient and appropriate method known in the art for the biomaterial.

The incubation time for the pathogen inactivating agent/biological material will depend largely on the identity and properties of the pathogen inactivating agent. Generally, incubation of biological materials, such a blood products, with the pathogen inactivating agent can be conducted for example, for about 5 minutes to 72 hours or more, or about 1 to 48 hours, for example, about 1 to 24 hours, or, for example, about 8 to 20 hours. For red blood cells, the incubation is typically conducted at a temperature of about 2° C. to 37° C., preferably about 18° C. to 25° C. For platelets, the temperature is preferably about 20° C. to 24° C. For plasma, the temperature may be about 0° C. to 60° C., typically about 0-24° C. Other acellular biological materials (e.g., purified proteins, tissue extracts, etc.) are normally incubated at about 0° C. to 25° C., generally at about 0° C. to about 10° C., most commonly at about 4° C.

Incubation may be with or without mixing, as desired. Typically, incubations of cellular materials, such as PRBC, will be carried out without mixing or with minimal mixing, to preserve the structural integrity of the cells in the biomaterial.

Preferably, inactivation of pathogens according to the instant methods accomplishes pathogen inactivation without damaging and/or modifying the biological material.

Where the biological material comprises RBCs, the lack of a substantially damaging effect on RBC function may be measured by methods known in the art for testing RBC function. For example, the levels of indicators such as intracellular ATP (adenosine 5′-triphosphate), intracellular 2,3-DPG (2,3-diphosphoglycerol) or extracellular potassium may be measured, and compared to an untreated control. Additionally hemolysis, pH, hematocrit, hemoglobin, osmotic fragility, glucose consumption and lactate production may be measured. Methods for determining ATP, 2,3-DPG, glucose, hemoglobin, hemolysis, and potassium are available in the art. See for example, Davey et al., Transfusion, 32:525-528 (1992), the disclosure of which is incorporated herein. Methods for determining red blood cell function are also described in Greenwalt et al., Vox Sang, 58:94-99 (1990); Hogman et al., Vox Sang, 65:271-278 (1993); and Beutler et al., Blood, Vol. 59 (1982) the disclosures of which are incorporated herein by reference. Extracellular potassium levels may be measured using a Ciba Corning Model 614 K+/Na+Analyzer (Ciba Corning Diagnostics Corp., Medford, Mass.). The pH can be measured using a Ciba Corning Model 238 Blood Gas Analyzer (Ciba Corning Diagnostics Corp., Medford, Mass.). Binding of species such as IgG, albumin, and IgM to red blood cells also may be measured using methods available in the art. Binding of molecules to red blood cells can be detected using antibodies, for example to acridine and IgG. Antibodies for use in assays can be obtained commercially, or can be made using methods available in the art, for example as described in Harlow and Lane, “Antibodies, a Laboratory Manual, Cold Spring Harbor Laboratory,” 1988, the disclosure of which is incorporated herein.

Use of the instant methods for pathogen inactivation of biological materials comprising RBCs (e.g., PRBC) preferably results in extracellular potassium levels not greater than 3 times, more preferably no more than 2 times the amount exhibited in an untreated control after 1 day. Hemolysis of biological materials containing RBCs is preferably less than 3% after 28 day storage, more preferably less than 2% after 42 day storage, and most preferably less than or equal to about 0.8% after 42 day storage at 4° C.

The lack of a substantially damaging effect on RBC function can also be assessed by looking at the in vivo survival of the red cells. Use of the instant methods for pathogen inactivation of biological materials comprising RBCs preferably results in greater than 75% survival after circulating 24 hours post transfusion into an appropriate model animal, such as a canine. More preferably, this 75% survival rate is maintained 24 hours post transfusion after storage of the treated red cells prior to transfusion for up to 7 days, 14 days, 21 days, 35 days, and 42 days at 4° C.

Biological materials such as acellular blood products, purified proteins, recombinant proteins and the like, when treated in accordance with the instant invention, preferably substantially retain the appropriate activity for their intended use(s), preferably at least 70%, 80%, 85%, 90%, 95% or 99% of pre-treatment activity. As will be apparent to one of skill in the art, the activity will vary depending on the exact identity of the biological material. For non-enzymatic, soluble biological materials such as albumin, immunoglobin, fibrinogen, and the like, the biological material remains substantially soluble (i.e., is at least 70%, 80%, 85%, 90%, 95% or 99% soluble compared to the material prior to treatment). Where the biological materials are enzymes, the biological materials retain substantially all of their enzymatic activity (i.e., is at least 70%, 80%, 85%, 90%, 95% or 99% activity compared to the material prior to treatment). Where the biological materials are cytokines, antibodies, growth factors, hormones, growth factor, cytokine or hormone-containing extracts, or other biological materials which rely upon specific receptor or antigen binding to exhibit biological activity, the biological materials preferably retain substantially all of their biological activity as compared to before treatment (i.e., are capable of at least 70%, 80%, 85%, 90%, 95% or 99% of pre-treatment binding to the appropriate receptor, or, alternatively, evoke 70%, 80%, 85%, 90%, 95% or 99% of the appropriate pre-treatment biological response in a target cell or tissue).

### EXAMPLES

### Example 1

### Y. enterocolitica Inactivation in Acellular Solutions

A series of experiments were carried out comparing additive solutions based on a slightly hypertonic, high chloride additive solution (additive A1) with additive solutions based on a hypotonic, low chloride additive solution (e.g. additive E1 in table 2) for pathogen inactivation.

4.2 milliliter (ml) aliquots of test additive solutions were dispensed into bacteriology culture tubes, spiked with the Gram negative bacteria Yersinia enterocolitica in 0.5 ml Luria broth (LB), then spiked with 0.33 ml of inactivation compound solution and incubated for two hours at room temperature (RT, about 19-26° C.), then assayed for bacterial titer. The 4.2 ml of additive A1 contained 2.5 mM adenine, 51.5 mM mannitol, and the sodium chloride concentrations indicated in table 2. The additive E1 contained 26.6 mM sodium citrate, 17.0 mM dibasic sodium phosphate, 4.7 mM monobasic sodium phosphate, 1.6 mM adenine, 42.5 mM mannitol and the sodium chloride concentrations indicated in table 2. The 0.33 ml of inactivation compound solution contained 30 mM glutathione and 3 mM β-alanine, N-(acridin-9-yl), 2-[bis(2-chloroethyl)amino]ethyl ester in either 10.05% dextrose (additive A1 samples) or 4.5% dextrose (additive E1 samples). This resulted in final concentrations of 2 mM glutathione, 0.2 mM inactivation compound, and 37 mM or 15 mM dextrose (additives A1 or E1, respectively). Bacterial titer was determined by plating a series of ten-fold dilutions of each sample and counting colonies. Pathogen inactivation was expressed as the base 10 log of the ratio of the bacterial titer of control to titer of the inactivated sample, or “log inactivation”. Results are summarized in Table 2.

Inactivation of Y. enterocolitica using additive A1 was poor (0.89 log inactivation), especially when compared to pathogen inactivation with Additive E1 instead of additive A1, which was complete (7.48 log inactivation, no detectable bacteria remaining). Reduction of NaCl concentration in additive A1 resulted in progressively higher log inactivation. A four fold reduction of the NaCl concentration in additive A1 resulted in a 0.42 log increase in inactivation(1.31-0.89). On the other hand addition of NaCl to additive E1 resulted in higher amounts of bacteria remaining (less inactivation). Notice that additive E1 150 solution, which has nearly the NaCl concentration of Adsol, had a 4.4 log reduction in inactivation compared to additive E1.

TABLE 2
Observed log inactivation for various test solutions.

| | mM sodium chloride | Log inactivation observed |
|---|---|---|
| Additive A1 | 192.5 | 0.89 |
| 0.5 additive A1 | 96.25 | 0.93 |
| 0.25 additive A1 | 48.06 | 1.31 |
| Additive E1 | 0 | 7.84 |
| Additive E1 10 | 10 | 5.63 |
| Additive E1 50 | 50 | 4.62 |
| Additive E1 100 | 100 | 3.81 |
| Additive E1 150 | 150 | 3.45 |

### Example 2

### Y. enterocolitica Inactivation in PRBC

Additive A1 and additive E1-based solutions were tested in combination with β-alanine, N-(acridin-9-yl), 2-[bis(2-chloroethyl)amino]ethyl ester plus glutathione for efficacy in pathogen inactivation in a cellular blood product, packed red blood cells (PRBC).

PRBC were prepared from whole blood by centrifuging the blood, then removing the supernatant plasma and anticoagulant. PRBC samples were then spiked with bacteria, mixed, and dispensed in 3.1 ml aliquots into bacteriological tubes. 1.55 ml of test additive solution at the concentrations indicated in Example 1 was added to the spiked PRBC, then 0.33 ml of dextrose solution containing 30 mM glutathione and 3 mM P-alanine, N-(acridin-9-yl), 2-[bis(2-chloroethyl)amino]ethyl ester was added, resulting in 0.2 mM inactivation compound and 2 mM glutathione with final dextrose concentrations as per Example 1. The samples were incubated two hours at room temperature, then assayed for bacterial titer.

The results are outlined in the Table 3. Log inactivation using additive E1 was high (4.84) but in additive A1 the inactivation was ˜3.8 logs less (1.08). As was found for acellular samples, altering NaCl concentration resulted in changes in pathogen inactivation. Decreased NaCl concentration in additive A1-based solutions resulted in progressively higher log inactivation. A four fold reduction of the NaCl concentration resulted in a 0.70 log increase in inactivation (i.e., 1.78-1.08). On the other hand, addition of NaCl to additive E1 resulted in reduced pathogen inactivation. Notice that when the NaCl concentration in additive E1 reached 150 mM (near the NaCl concentration in additive A1), the inactivation was reduced by 3 logs, close to the levels of the additive A1 solution. The differences in inactivation observed in this example compared to the previous example (no red cells) may be attributed to ions and metabolites contributed by the red cells, which occupied a significant portion of the volume. The presence of leukocytes in the samples containing red cells may also account for some of the differences, as they may interfere with the bacterial inactivation.

TABLE 3
Observed log inactivation in red cells containing
various test solutions.

| Additive Solution | Log Inactivation |
|---|---|
| Additive A1 | 1.08 |
| 0.5 additive A1 | 1.90 |
| 0.25 additive A1 | 1.78 |
| Additive E1 | 4.83 |
| Additive E1 10 | 2.39 |
| Additive E1 50 | 2.36 |
| Additive E1 100 | 2.08 |
| Additive E1 150 | 1.95 |

### Example 3

### Pathogen Inactivation in PRBC

Pathogen inactivation was tested using a variety of Gram negative and Gram positive bacterial pathogens. Full PRBC units (300 ml, in oxygen permeable containers) were spiked with Gram negative pathogens Serratia marcescens, Pseudomonas fluorescens, Salmonella typhymurium, or Y. enterocolitica or Gram positive pathogens Staphylococcus aureus or Staphylococcus epiderimidis. The spiked PRBC units were mixed with Adsol, additive E1, or Solution 2 (Gram negative bacteria only) formulations lacking glucose, then βalanine, N-(acridin-9-yl), 2-[bis(2-chloroethyl)amino]ethyl ester and glutathione in dextrose (10.05% for Adsol and solution 2, 4.5% for additive E1) was added. Adsol, and Solution 2 formulations are shown in Table 1. Additive E1 is as per Examples 1 and 2. β-alanine, N-(acridin-9-yl), 2-[bis(2-chloroethyl)amino]ethyl ester and glutathione were added to a final concentration of 0.2 mM and 2 mM, respectively. Each sample was incubated for two hours at RT, then assayed for bacterial titer and inactivation.

Additive E1 and Solution 2, both chloride-free, hypotonic solutions, gave about equivalent log inactivation when both additive solutions were tested on a given Gram negative pathogen. The log inactivation for additive E1 and/or Solution 2 was consistently greater than for Adsol in all Gram negative strains. For Gram positive strains, additive E1 was compared to Adsol and showed improved inactivation in only the Staphylococcus epidermidis. Results are shown in Tables 4 and 5.

TABLE 4

| | Gram Negative Pathogens Log inactivation | | | |
|---|---|---|---|---|
| | Y. | P. | S. | S. |
| Solution | enterocolitica | fluorescens | marcescens | typhymurium |
| Adsol | 2.47 | 3.86 | nd | 1.31 |
| Erythrosol | 4.2 | 4.6 | 4.17 | 4.11 |
| Solution 2 | 4.35 | 4.25 | 4.24 | nd |

TABLE 5

Gram Positive Pathogens Log inactivation

| Solution | S. aureus | S. epidermidis |
|---|---|---|
| Adsol | 4.8 | 4.27 |
| Erythrosol | 4.25 | 6.33 |

### Example 4

### Pathogen Inactivation Processing with Various Additive Solutions

The inactivation of pathogens using nucleic acid targeted effector compounds is done using a variety of additive solutions. Typically, about 450 ml of whole blood is collected into a bag containing 63 ml of CPD. The red cells are concentrated by centrifuging at 4100×g for about 5 minutes and the plasma fraction is removed, leaving an about 200 ml volume of concentrated red cells. Following this, about 100 to 120 ml of the desired additive solution is added (typically as follows; 100 ml for Nutricel, Erythrosol or SAG-M, 110 ml for Adsol or solution 2, and 114 ml for E2 or E3, see table 6). The pathogen inactivation compound, typically in a solid form, is dissolved in the additive solution at this point and added to the red cells along with the additive solution. The red cell solution is then incubated at room temperature for sufficient times to effect the inactivation of any pathogen that may be present. In some instances, for example using Erythrosol, the dextrose component of the additive solution is separate (part B) from the remaining components (part A) and the pathogen inactivation compound is dissolved in the dextrose solution for addition to the red cells. Alternatively, the pathogen inactivation compound is added independently from the additive solution or the dextrose. The concentrations in Table 1 for Erythrosol and solution 2 are for the combined parts A and B. For Erythrosol, part A is typically 94 ml and part B is typically 6 ml. For solution 2, part A is typically 90 ml and part B is typically 20 ml. The concentrations of other suitable additives are given in Table 6. Additives E2 and E3 may also have the dextrose added separately (part B) where the concentrations given are for the combined parts A and B and part A is typically 94 ml and part B is 20 ml.

TABLE 6
Red cell additive solutions.

| | Concentration of components (mM) | | | | | | |
|---|---|---|---|---|---|---|---|
| Additive Solution | Na₃citrate | Dextrose | NaH₂PO₄ | Na₂HPO₄ | Adenine | Mannitol | NaCl |
| Nutricel | | 55.5 | 23.0 | | 2.2 | | 70.0 |
| Optisol | | 45.4 | | | 2.2 | 45.4 | 150.0 |
| SAG-M | | 45.4 | | | 1.3 | 28.8 | 150.1 |
| E2 | 21.9 | 39.8 | 3.9 | 14.0 | 1.3 | 35.0 | |
| E3 | 21.9 | 70.8 | 3.9 | 14.0 | 1.3 | 35.0 | |

The above process is used to evaluate the efficacy of the inactivation process. In this case, known amounts of a suitable pathogen are added following the removal of plasma from the centrifuged red cells. The level of inactivation is compared to a control solution which does not contain the pathogen inactivation compound. The log inactivation is determined by assessing the bacterial titer of inactivated sample as compared to control per example 1.

### Example 5

### Inactivation of Pathogens using Erythrosol Additive Solution.

Pathogen inactivation was demonstrated using a variety of Gram negative and Gram positive bacterial pathogens as well as a variety of viral pathogens. Leukoreduced full PRBC units (300 ml, in oxygen permeable containers) were spiked with Gram negative pathogens Serratia marcescens, Salmonella choleraesuis, Escherichia coli K12, Pseudomonas aeruginosa, Serratia liquifaciens, or Y. enterocolitica, Gram positive pathogens Staphylococcus aureus, Staphylococcus epidermidis, Deinococcus radiodurans, Listeria monocytogenes, or Bacilus subtilis, or viral pathogens Human Immunodeficiency Virus (HIV, both cell-free and cell-associated), Duck Hepatitis B Virus (DHBV), Bovine Viral Diarrhea Virus (BVDV), Herpes Simplex Virus (HSV), Respiratory Synctial Virus (RSV), Vesicular Stomatitis Virus type Indiana (VSIV) or Bluetongue type 11 Virus. The spiked PRBC units were mixed with Erythrosol, then β-alanine, N-(acridin-9-yl), 2-[bis(2-chloroethyl)amino]ethyl ester and glutathione in dextrose was added. β-alanine, N-(acridin-9-yl), 2-[bis(2-chloroethyl)amino]ethyl ester and glutathione were added to a final concentration as indicated in Table 7A-B. Each sample was incubated for two hours at RT, then assayed for bacterial or viral titer and inactivation.

The results indicated the variety of bacterial and viral pathogens that are inactivated using Erythrosol, an additive solution of the present invention.

TABLE 7A
Levels of inactivation of various viral pathogens in red cells using
Erythrosol additive solution and β-alanine, N-(acridin-9-yl),
2-[bis(2-chloroethyl)amino]ethyl ester (inactivation compound).

| | mM inactivation | | |
|---|---|---|---|
| Virus | compound | mM glutathione | Log inactivation |
| Cell-Free HIV | 0.2 | 3 | >6.5 |
| Cell-Associated | 0.2 | 3 | >6.2 |
| HIV | | | |
| DHBV | 0.1 | 3 | >6.3 |
| HSV | 0.003 | 3 | >6.0 |
| BVDV | 0.1 | 1 | >7.3 |
| RSV | 0.2 | 2 | 5.6 |
| VSIV | 0.2 | 2 | 5.7 |
| Bluetongue | 0.2 | 2 | 6.0 |
| type 11 | | | |

TABLE 7B
Levels of inactivation of various bacterial pathogens in red
cells using Erythrosol additive solution and β-alanine,
N-(acridin-9-yl), 2-[bis(2-chloroethyl)amino]ethyl ester.
Reaction conditions are 0.2 mM β-alanine, N-(acridin-9-yl),
2-[bis(2-chloroethyl)amino]ethyl ester and 2 mM glutathione.

| Bacteria | Gram stain | Log inactivation |
|---|---|---|
| Yersinia enterocolitica | negative | 7.4 |
| Serratia marcescens | negative | 4.1 |
| Eseherichia coli K12 | negative | 7.4 |
| Salmonella choleraesuis | negative | 4.8 |
| Pseudomonas aeruginosa | negative | 4.5 |
| Serratia liquifaciens | negative | 3.8 |
| Staphylococcus epidermidis | positive | >6.9 |
| Staphylococcus aureus | positive | >5.1 |
| Deinococcus radiodurans | positive | >6.0 |
| Listeria monocytogenes | positive | >7.1 |
| Bacilus subtilis | positive | >6.3 |

We claim:

1. An aqueous mixture for inactivation of bacteria comprising:
(i) an additive solution wherein chloride ion, if present, is at a concentration of less than about 10 mM,
(ii) a biological material suspected of containing the bacteria; and
(iii) a pathogen inactivation compound in an amount sufficient to inactivate at least 1 log of the bacteria.

2. The aqueous mixture of claim 1 wherein the additive solution is essentially free of chloride ions.

3. The aqueous mixture of claim 1 wherein the additive solution is hypotonic.

4. The aqueous mixture of claim 1 wherein the bacteria is selected from the group consisting of Yersinia enterocolitica, Pseudomonas fluorescens, Serratia marcescens, Salmonella Typhymurium, Salmonella choleraesuis, Escherichia coli K12, Pseudomonas aeruginosa, Serratia liquifaciens, and Staphylococcus epidermidis.

5. The aqueous mixture of claim 1 wherein the bacteria is a Gram negative bacteria.

6. The aqueous mixture of claim 5-wherein the Gram negative bacteria is selected from the group consisting of Yersinia enterocolitica, Pseudomonas fluorescens, Serratia marcescens, Salmonella Typhymurium Salmonella choleraesuis, Escherichia coli K12, Pseudomonas aeruginosa, and Serratia liquifaciens.

7. The aqueous mixture of claim 6 wherein the Gram negative bacteria is selected from the group consisting of Yersinia enterocolitica, Pseudomonas fluorescens, Serratia marcescens, and Salmonella Typhymurium.

8. The aqueous mixture of claim 1 wherein the biological material comprises a blood product.

9. The aqueous mixture of claim 8 wherein the blood product further comprises red blood cells.

10. The aqueous mixture of claim 1 wherein the pathogen inactivation compound is more reactive at physiological pH than at a pH of about 4.

11. The aqueous mixture of claim 1 wherein the pathogen inactivation compound in the aqueous mixture is at a concentration of between about 0.1 μM to about 5 mM.

12. The aqueous mixture of claim 11 wherein the pathogen inactivation compound is at a concentration of between about 10 μM to about 750 μM.

13. A method of inactivating a bacteria in a biological material suspected of containing the bacteria comprising:
(i) contacting the biological material with an additive solution comprising a chloride concentration of less than about 10 mM,
(ii) contacting the biological material with a pathogen inactivation compound in an amount sufficient to inactivate at least 1 log of the bacteria, and
(iii) incubating the biological material contacted with the additive solution and the pathogen inactivation compound for sufficient time to inactivate at least 1 log of the bacteria.

14. The method of claim 13 wherein the additive solution is essentially free of chloride ions.

15. The method of claim 13 wherein the additive solution is hypotonic.

16. The method of claim 13 wherein the bacteria is selected from the group consisting of Yersinia enterocolitica, Pseudomonas fluorescens, Serratia marcescens, Salmonella Typhymurium Salmonella choleraesuis, Escherichia coli K12, Pseudomonas aeruginosa, and Serratia liquifaciens.

17. The method of claim 13 wherein the bacteria is a Gram negative bacteria.

18. The method of claim 17 wherein the Gram negative bacteria is selected from the group consisting of Yersinia enterocolitica, Pseudomonas fluorescens, Serratia marcescens, Salmonella Typhymurium Salmonella choleraesuis, Escherichia coli K12, Pseudomonas aeruginosa, and Serratia liquifaciens.

19. The method of claim 18 wherein the Gram negative bacteria is selected from the group consisting of Yersinia enterocolitica, Pseudomonas fluorescens, Serratia marcescens, and Salmonella Typhymurium.

20. The method of claim 13 wherein the biological material comprises a blood product.

21. The method of claim 20 wherein the blood product further comprises red blood cells.

22. The method of claim 13 wherein the pathogen inactivation compound is more reactive at physiological pH than at a pH of about 4.

23. The method of claim 13 wherein the pathogen inactivation compound is at a concentration of between about 0.1 μM to about 5 mM at the beginning of said incubation.

24. The method of claim 23 where in the pathogen inactivation compound is at a concentration of between about 10 μM to about 750 μM at the beginning of said incubation.

25. The method of claim 13 wherein the incubation is carried out at a temperature of between about 18° C. to 25° C.

26. The method of claim 25 wherein the incubation is carried out for between about 1 hour to about 48 hours.

27. A method of inactivating a bacteria in a biological material suspected of containing the bacteria comprising:
(i) contacting the biological material with a first additive solution which is essentially chloride free,
(ii) contacting the biological material with a pathogen inactivation compound in an amount sufficient to inactivate at least 1 log of the bacteria, wherein the pathogen inactivation compound has a greater inactivation efficiency against Yersinia enterocolitica when used with said first additive solution than when used with a second additive solution, said second additive solution comprising at least about 10 mM chloride ion; and
(iii) incubating the biological material contacted with the first additive solution and the pathogen inactivation compound for sufficient time to inactivate at least 1 log of the bacteria.

28. The method of claim 27 wherein the additive solution is hypotonic.

29. The method of claim 27 wherein the bacteria is selected from the group consisting of Yersinia enterocolitica, Pseudomonas fluorescens, Serratia marcescens, Salmonella Typhymurium Salmonella choleraesuis, Escherichia coli K12, Pseudomonas aeruginosa, and Serratia liquifaciens.

30. The method of claim 27 wherein the bacteria is a Gram negative bacteria.

31. The method of claim 30 wherein the Gram negative bacteria is selected from the group consisting of Yersinia enterocolitica, Pseudomonas fluorescens, Serratia marcescens, Salmonella Typhymurium Salmonella choleraesuis, Escherichia coli K12, Pseudomonas aeruginosa, and Serratia liquifaciens.

32. The method of claim 31 wherein the Gram negative bacteria is selected from the group consisting of Yersinia enterocolitica, Pseudomonas fluorescens, Serratia marcescens, and Salmonella Typhymurium.

33. The method of claim 27 wherein the biological material comprises a blood product.

34. The method of claim 33 wherein the blood product further comprises red blood cells.

35. The method of claim 27 wherein the pathogen inactivation compound is more reactive at physiological pH than at a pH of about 4.

36. The method of claim 27 wherein the pathogen inactivation compound is at a concentration of between about 0.1 μM to about 5 mM at the beginning of said incubation.

37. The method of claim 36 wherein the pathogen inactivation compound is at a concentration of between about 10 μM to about 750 μM at the beginning of said incubation.

38. The method of claim 27 wherein the incubation is carried out at a temperature of between about 18° C. to 25° C.

39. The method of claim 38 wherein the incubation is carried out for between about 1 hour to about 48 hours.

40. A method of inactivating a bacteria in a biological material suspected of containing the bacteria comprising:
(i) contacting the biological material with a pathogen inactivation compound in an amount sufficient to inactivate at least 1 log of the bacteria and an additive solution comprising a chloride concentration of less than about 10 mM, and
(ii) incubating the biological material contacted with the additive solution and the pathogen inactivation compound for sufficient time to inactivate at least I log of the bacteria.

41. The method of claim 40 wherein the additive solution is essentially free of chloride ions.

42. The method of claim 40 wherein the additive solution is hypotonic.

43. The method of claim 40 wherein the bacteria is selected from the group consisting of Yersinia enterocolitica, Pseudomonas fluorescens, Serratia marcescens, Salmonella Typhymurium Salmonella choleraesuis, Escherichia coli K12, Pseudomonas aeruginosa, and Serratia liquifaciens.

44. The method of claim 40 wherein the bacteria is a Gram negative bacteria.

45. The method of claim 44 wherein the Gram negative bacteria is selected from the group consisting of Yersinia enterocolitica, Pseudomonas fluorescens, Serratia marcescens, Salmonella Typhymurium Salmonella choleraesuis, Escherichia coli K12, Pseudomonas aeruginosa, and Serratia liquifaciens.

46. The method of claim 45 wherein the Gram negative bacteria is selected from the group consisting of Yersinia enterocolitica, Pseudomonas fluorescens, Serratia marcescens, and Salmonella Typhymurium.

47. The method of claim 40 wherein the biological material comprises a blood product.

48. The method of claim 47 wherein the blood product further comprises red blood cells.

49. The method of claim 40 wherein the pathogen inactivation compound is more reactive at physiological pH than at a pH of about 4.

50. The method of claim 40 wherein the pathogen inactivation compound is at a concentration of between about 0.1 μM to about 5 mM at the beginning of said incubation.

51. The method of claim 50 where in the pathogen inactivation compound is at a concentration of between about 10 μM to about 750 μM at the beginning of said incubation.

52. The method of claim 40 wherein the incubation is carried out at a temperature of between about 18° C. to 25° C.

53. The method of claim 52 wherein the incubation is carried out for between about 1 hour to about 48 hours.

54. A method of inactivating a bacteria in a biological material suspected of containing the Gram negative bacteria comprising:
(i) contacting the biological material with a first additive solution which is essentially chloride free and a pathogen inactivation compound in an amount sufficient to inactivate at least 1 log of the bacteria, wherein the pathogen inactivation compound has a greater inactivation efficiency against Yersinia enterocolitica when used with said first additive solution than when used with a second additive solution, said second additive solution comprising at least about 10 mM chloride ion; and
(iii) incubating the biological material contacted with the first additive solution and the pathogen inactivation compound for sufficient time to inactivate at least 1 log of the bacteria.

55. The method of claim 54 wherein the additive solution is hypotonic.

56. The method of claim 54 wherein the bacteria is selected from the group consisting of Yersinia enterocolitica, Pseudomonas fluorescens, Serratia marcescens, Salmonella Typhymurium Salmonella choleraesuis, Escherichia coli K12, Pseudomonas aeruginosa, and Serratia liquifaciens.

57. The method of claim 54 wherein the bacteria is a Gram negative bacteria.

58. The method of claim 57 wherein the Gram negative bacteria is selected from the group consisting of Yersinia enterocolitica, Pseudomonas fluorescens, Serratia marcescens, Salmonella Typhymurium Salmonella choleraesuis, Escherichia coli K12, Pseudomonas aeruginosa, and Serratia liquifaciens.

59. The method of claim 58 wherein the Gram negative bacteria is selected from the group consisting of Yersinia enterocolitica, Pseudomonas fluorescens, Serratia marcescens, and Salmonella Typhymurium.

60. The method of claim 54 wherein the biological material comprises a blood product.

61. The method of claim 60 wherein the blood product further comprises red blood cells.

62. The method of claim 54 wherein the pathogen inactivation compound is more reactive at physiological pH than at a pH of about 4.

63. The method of claim 54 wherein the pathogen inactivation compound is at a concentration of between about 0.1 μM to about 5 mM at the beginning of said incubation.

64. The method of claim 63 where in the pathogen inactivation compound is at a concentration of between about 10 μM to about 750 μM at the beginning of said incubation.

65. The method of claim 54 wherein the incubation is carried out at a temperature of between about 18° C. to 25° C.

66. The method of claim 65 wherein the incubation is carried out for between about 1 hour to about 48 hours.

67. A method of inactivating a bacteria in a biological material suspected of containing the bacteria comprising:
(i) contacting the biological material with an additive solution that is essentially free of chloride ions and comprises about 26.6 mM sodium citrate, about 17 mM disodium phosphate, about 4.7 mM monosodium phosphate, about 1.6 mM adenine and about 42.5 mM mannitol,
(ii) contacting the biological material with a pathogen inactivation compound in an amount sufficient to inactivate at least 1 log of the bacteria, and
(iii) incubating the biological material contacted with the additive solution and the pathogen inactivation compound for sufficient time to inactivate at least 1 log of the bacteria.
